(19) United States
(12) Patent Application Publication
(10) Pub. No.: US 2026/0077182 A1
(43) Pub. Date: Mar. 19, 2026

(54) ELECTROTHERAPY DEVICE

(71) Applicant: OMRON HEALTHCARE Co., Ltd., Kyoto (JP)

(72) Inventors: Yoshiyuki TSUJI, Kyoto (JP); Tomoyuki NISHIDA, Kyoto (JP)

(21) Appl. No.: 19/395,676

(22) Filed: Nov. 20, 2025

Related U.S. Application Data
(63) Continuation of application No. PCT/JP2024/002350, filed on Jan. 26, 2024.

(30) Foreign Application Priority Data
Sep. 12, 2023 (JP) 2023-147324

(51) Int. Cl. A61N 1/04

(52) U.S. Cl. CPC A61N 1/0492

(57) ABSTRACT

An electrotherapy device includes an electrode portion having an adhesive portion, a non-adhesive portion provided so as to be capable of being in contact with a body surface, and an operation portion configured to switch from a state in which the adhesive portion is not in contact with the body surface to a state in which the adhesive portion is in contact with the body surface. The adhesive portion abuts the body surface, by operating the operation portion in an abutting state in which the non-adhesive portion abuts the body surface such that the adhesive portion is not in contact with the body surface.

### CROSS-REFERENCE TO RELATED APPLICATIONS

This application is the U.S. national stage application filed pursuant to 35 U.S.C. 365(c) and 120 as a continuation of International Patent Application No. PCT/JP2024/002350, filed January 26, 2024, which application claims priority to Japanese Patent Application No. 2023-147324, filed September 12, 2023, which applications are incorporated herein by reference in their entireties.

### FIELD

The present disclosure relates to an electrotherapy device.

### BACKGROUND

In the related art, as an electrotherapy device such as a low-frequency treatment device, JP 2017-047065 A (Patent Document 1) discloses a configuration including a pad portion that is attached to a body surface of a user to apply a low-frequency pulse current to the user, and a main body portion that supplies the low-frequency pulse current to the pad portion.

### SUMMARY

### Technical Problem

The pad portion (electrode portion) serving as a conductive portion of the electrotherapy device needs to be fixed to the body surface of the user, and thus includes an adhesive portion such as a hydrogel or an adhesive sheet. In a case where such an adhesive portion is provided, it is not possible to position the pad portion while shifting the position of the pad portion. Therefore, it is often the case that the pad portion cannot be attached successfully to an affected site to which the pad portion is desired to be attached in the lower body, a back portion, or the like where it is difficult to reach with a hand or to visually confirm.

In addition, in a structure in which the main body portion and the pad portion including the adhesive portion are integrated, it is necessary to simultaneously perform positioning and attachment of the pad portion while holding the main body portion. In such a case, the adhesive portion is frequently attached to an unintended place, and thus the adhesive portion is frequently peeled off and attached again.

The present disclosure has been made in view of the above problems, and an object of the present disclosure is to provide an electrotherapy device capable of accurately attaching an adhesive portion provided at an electrode portion to a desired treatment site with a simple operation.

### Solution To Problem

An electrotherapy device according to the present disclosure includes: an electrode portion including an adhesive portion; a non-adhesive portion provided so as to be capable of being in contact with a body surface, and an operation portion configured to switch from a state in which the adhesive portion is not in contact with the body surface to a state in which the adhesive portion is in contact with the body surface. The adhesive portion abuts the body surface, by operating the operation portion in an abutting state in which the non-adhesive portion abuts the body surface such that the adhesive portion is not in contact with the body surface.

The electrotherapy device according to the present disclosure may further include a holding portion that is movably provided and holds the electrode portion. The holding portion may move and the adhesive portion may abut the body surface, by operating the operation portion.

In the electrotherapy device according to the present disclosure, the non-adhesive portion may movably support the holding portion.

In the electrotherapy device according to the present disclosure, the operation portion may be coupled to the holding portion. In this case, the holding portion may move and the adhesive portion may abut the body surface, by pressing the operation portion.

In the electrotherapy device according to the present disclosure, the holding portion may include a first surface facing the body surface in the abutting state and a second surface located on a side opposite to the first surface. The electrode portion may be provided at the first surface. The operation portion may be provided at the second surface.

In the electrotherapy device according to the present disclosure, the non-adhesive portion may rotatably support the holding portion. The holding portion includes one end and another end. The electrode portion may be provided on the one end side of the holding portion, and the operation portion may be provided on the other end side of the holding portion. In the abutting state in which the non-adhesive portion abuts the body surface such that the adhesive portion is not in contact with the body surface, the one end side of the holding portion may be farther from the body surface than the other end side of the holding portion is. In this case, the adhesive portion may abut the body surface, by rotating the holding portion from the abutting state such that the one end side of the holding portion approaches the body surface.

The electrotherapy device according to the present disclosure may include a drive unit configured to move the holding portion, and a control unit configured to control an operation of the drive unit. An operation signal may be input to the control unit, by operating the operation portion. The holding portion may move such that the adhesive portion approaches the body surface, through the control unit may controlling the operation of the drive unit based on the operation signal.

The electrotherapy device according to the present disclosure may include an operation terminal provided with the operation portion. The operation terminal may include a transmission unit configured to transmit an operation signal to the control unit.

In the electrotherapy device according to the present disclosure, the holding portion may be provided so as to be elastically deformable. The electrotherapy device may further include a lock mechanism that releasably maintains a state in which the holding portion is elastically deformed so as to exert an elastic force. The operation portion may be provided so as to be capable of releasing the lock mechanism. In this case, the holding portion may be moved by a restoring force based on the elastic force and the adhesive portion may abut the body surface, by releasing the lock mechanism releasing via the operation portion.

The electrotherapy device according to the present disclosure may further include an interposed member interposed between the adhesive portion and the body surface in the abutting state, and a retracting portion provided to be capable of retracting the interposed member. In this case, the interposed member may be retracted by the retracting portion and the adhesive portion may abut the body surface, by operating the operation portion.

In the electrotherapy device according to the present disclosure, the non-adhesive portion may be a main body portion that supplies current to the electrode portion.

In the electrotherapy device according to the present disclosure, the operation portion may be provided so as to be operable as a power switch of the main body portion.

### Advantageous Effects of Invention

According to the present disclosure, it is possible to provide an electrotherapy device capable of accurately attaching an adhesive portion provided at an electrode portion to a desired treatment site with a simple operation.

### BRIEF DESCRIPTION

Various embodiments are disclosed, by way of example only, with reference to the accompanying schematic drawings in which corresponding reference symbols indicate corresponding parts, in which:

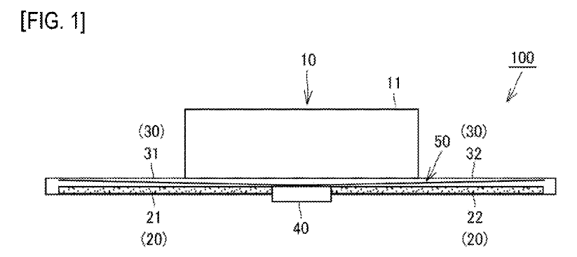

FIG. 1 is a schematic diagram of an electrotherapy device according to Embodiment 1.

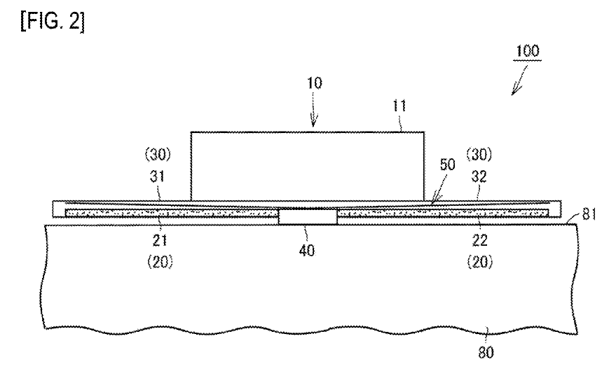

FIG. 2 is a diagram illustrating a state in which the electrotherapy device according to Embodiment 1 is positioned at a body surface of a user.

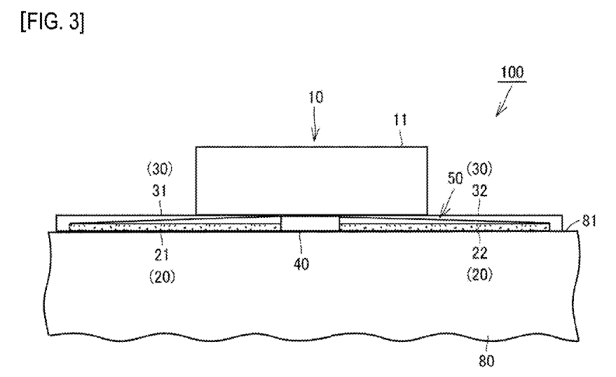

FIG. 3 is a diagram illustrating a state in which an electrode portion of the electrotherapy device according to Embodiment 1 is attached to the body surface of the user.

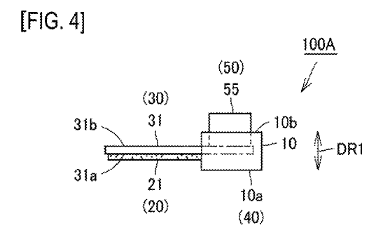

FIG. 4 is a schematic diagram of an electrotherapy device according to Embodiment 2.

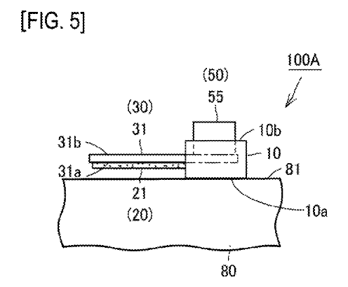

FIG. 5 is a diagram illustrating a state in which the electrotherapy device according to Embodiment 2 is positioned at a body surface of a user.

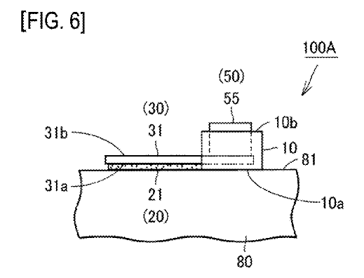

FIG. 6 is a diagram illustrating a state in which an electrode portion of the electrotherapy device according to Embodiment 2 is attached to the body surface of the user.

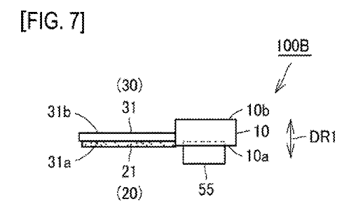

FIG. 7 is a schematic diagram of an electrotherapy device according to Embodiment 3.

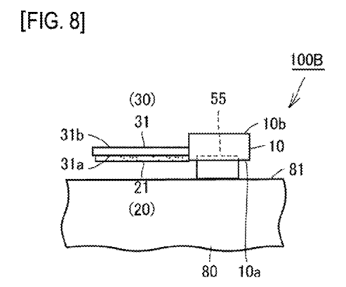

FIG. 8 is a diagram illustrating a state in which the electrotherapy device according to Embodiment 3 is positioned at a body surface of a user.

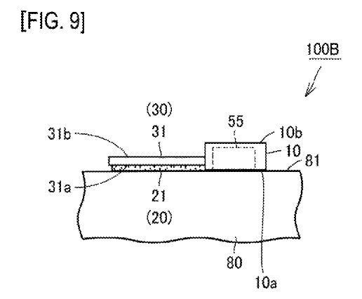

FIG. 9 is a diagram illustrating a state in which an electrode portion of the electrotherapy device according to Embodiment 3 is attached to the body surface of the user.

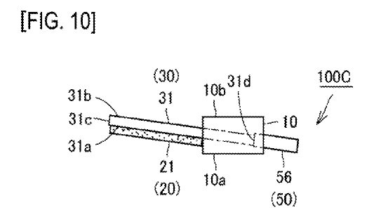

FIG. 10 is a schematic diagram of an electrotherapy device according to Embodiment 4.

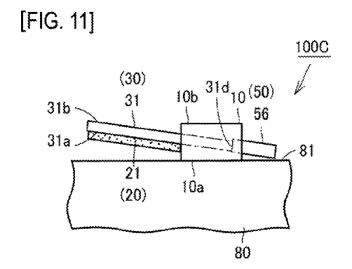

FIG. 11 is a diagram illustrating a state in which the electrotherapy device according to Embodiment 4 is positioned at a body surface of a user.

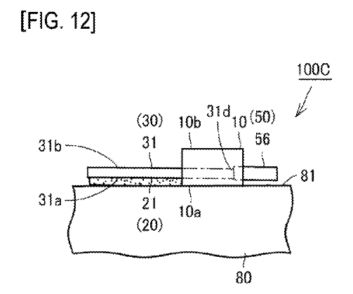

FIG. 12 is a diagram illustrating a state in which an electrode portion of the electrotherapy device according to Embodiment 4 is attached to the body surface of the user.

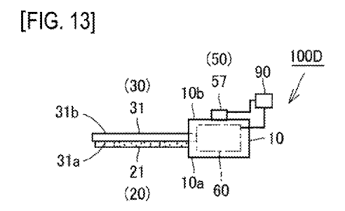

FIG. 13 is a schematic diagram of an electrotherapy device according to Embodiment 5.

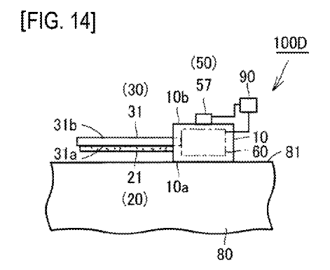

FIG. 14 is a diagram illustrating a state in which the electrotherapy device according to Embodiment 5 is positioned at a body surface of a user.

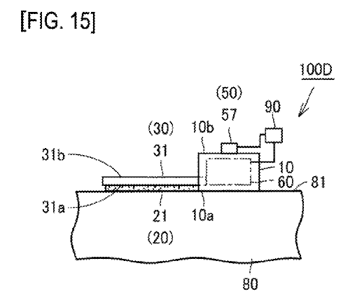

FIG. 15 is a diagram illustrating a state in which an electrode portion of the electrotherapy device according to Embodiment 5 is attached to the body surface of the user.

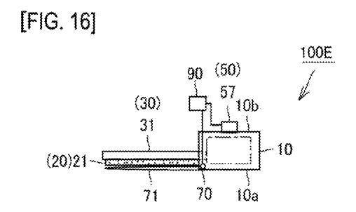

FIG. 16 is a schematic diagram of an electrotherapy device according to Embodiment 6.

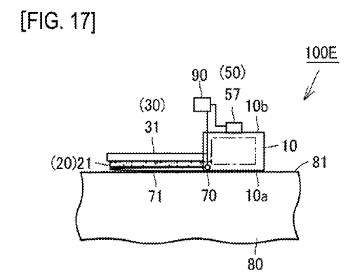

FIG. 17 is a diagram illustrating a state in which the electrotherapy device according to Embodiment 6 is positioned at a body surface of a user.

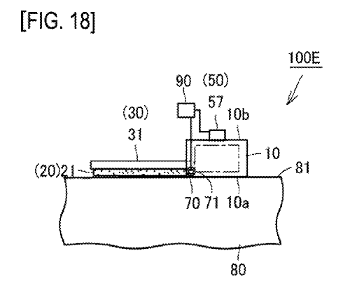

FIG. 18 is a diagram illustrating a state in which an electrode portion of the electrotherapy device according to Embodiment 6 is attached to the body surface of the user.

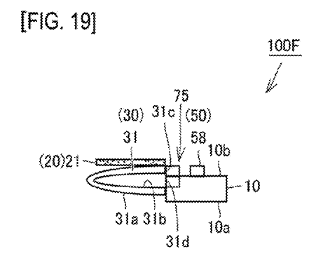

FIG. 19 is a schematic diagram of an electrotherapy device according to Embodiment 7.

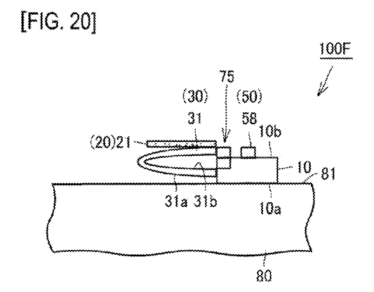

FIG. 20 is a diagram illustrating a state in which an electrotherapy device according to Embodiment 7 is positioned at a body surface of a user.

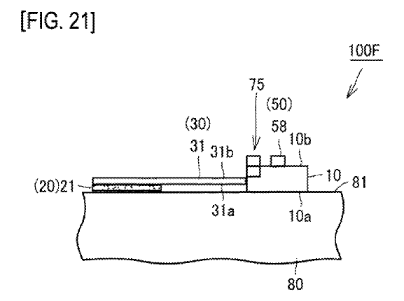

FIG. 21 is a diagram illustrating a state in which an electrode portion of the electrotherapy device according to Embodiment 7 is attached to the body surface of the user.

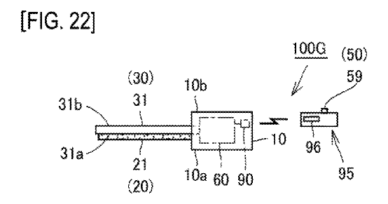

FIG. 22 is a schematic diagram of an electrotherapy device according to Embodiment 8.

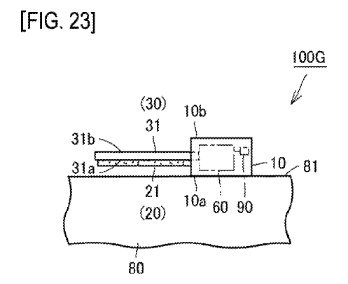

FIG. 23 is a diagram illustrating a state in which the electrotherapy device according to Embodiment 8 is positioned at a body surface of a user.

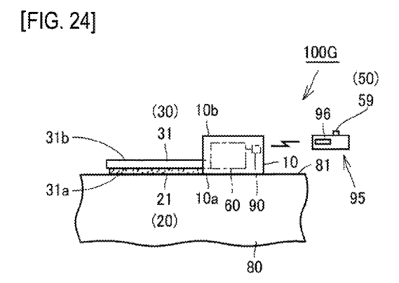

FIG. 24 is a diagram illustrating a state in which an electrode portion of the electrotherapy device according to Embodiment 8 is attached to the body surface of the user.

### DESCRIPTION OF EMBODIMENTS

Embodiments of the present disclosure will be described below with reference to the drawings. In the embodiments described below, the same or common components are denoted by the same reference signs in the drawings, and the descriptions thereof are not repeated.

Furthermore, it is understood that this disclosure is not limited to the particular methodology, materials and modifications described and as such may, of course, vary. It is also understood that the terminology used herein is for the purpose of describing particular aspects only, and is not intended to limit the scope of the claims. Those in the art will understand that any suitable material, now known, or hereafter developed, may be used in forming the present invention described herein.

Unless defined otherwise, all technical and scientific terms used herein have the same meaning as commonly understood to one of ordinary skill in the art to which this disclosure pertains. It should be understood that any methods, devices or materials similar or equivalent to those described herein can be used in the practice or testing of the example embodiments.

Broadly, and in view of all of the representative illustrations, the present disclosure provides an electrotherapy device 100A, which may include an electrode portion 20 including an adhesive portion; a non-adhesive portion provided so as to be capable of being in contact with a body surface 81; and an operation portion 50 configured to switch from a state in which the adhesive portion is not in contact with the body surface 81 to a state in which the adhesive portion is in contact with the body surface. The adhesive portion abuts the body surface 81, by operating the operation portion 50 in an abutting state in which the non-adhesive portion abuts the body surface such that the adhesive portion is not in contact with the body surface.

### Embodiment 1

FIG. 1 is a schematic diagram of an electrotherapy device according to Embodiment 1. An electrotherapy device 100 according to Embodiment 1 will be described with reference to FIG. 1.

As illustrated in FIG. 1, the electrotherapy device 100 according to Embodiment 1 includes a main body portion 10, an electrode portion 20, a holding portion 30, a non-adhesive portion 40, and an operation portion 50.

The electrotherapy device 100 is, for example, a low-frequency treatment device that alleviates a user's pain by supplying a low-frequency pulse current to the electrode portion 20 that is in contact with a body surface (see FIG. 2) of a user 80 (see FIG. 2). The frequency of the low-frequency pulse current is, for example, 1 Hz or higher to 1200 Hz or lower.

The main body portion 10 has a substantially box shape. The main body portion 10 supplies a current to the electrode portion 20. Specifically, the main body portion 10 includes a circuit unit (not illustrated) and a housing case 11. The housing case 11 accommodates therein the circuit unit. The circuit unit controls the current and the voltage flowing through the electrode portion 20 (a first electrode portion 21 and a second electrode portion 22) via an electric path.

The electrode portion 20 includes the first electrode portion 21 and the second electrode portion 22. The first electrode portion 21 and the second electrode portion 22 are electrode portions that cause a current to flow to the user and are provided so as to be attachable to a body surface 81. In a state in which the first electrode portion 21 and the second electrode portion 22 are in contact with the body surface of the user, a current is supplied to the first electrode portion 21 and the second electrode portion 22, thereby causing the current to flow to the user.

The first electrode portion 21 and the second electrode portion 22 may be, for example, gel pads or may be stacked bodies each constituted by an adhesive layer and an electrode portion. In a case where the first electrode portion 21 and the second electrode portion 22 are gel pads, the gel pads function as the adhesive portions. In a case where the first electrode portion 21 and the second electrode portion 22 each include an adhesive layer and an electrode portion, the adhesive layer functions as the adhesive portion.

The holding portion 30 holds the electrode portion 20. The holding portion 30 includes a first holding portion 31 and a second holding portion 32. The holding portion 30 is provided so as to allow movement of the non-adhesive portion 40 and deformation of the operation portion 50 as will be described below.

The non-adhesive portion 40 is disposed so as to overlap with the main body portion 10. The non-adhesive portion 40 is disposed between the first electrode portion 21 and the second electrode portion 22. The non-adhesive portion 40 is provided so as to protrude toward the body surface 81 side from the electrode portion 20 in a state in which the electrotherapy device 100 is disposed separately from the body surface 81 such that the electrode portion 20 faces the body surface 81. The non-adhesive portion 40 is provided so as to be in contact with the body surface 81 in a state in which the adhesive portion of the electrode portion 20 is not in contact with the body surface 81. The non-adhesive portion 40 is formed of a resin member, for example.

The operation portion 50 switches from a non-contact state in which the adhesive portion is not in contact with the body surface 81 to a contact state in which the adhesive portion is in contact with the body surface 81. In Embodiment 1, the operation portion 50 is formed of a thin metal plate, for example. The operation portion 50 is provided such that a connection portion connected to the non-adhesive portion 40 is deformable between a state of being away from the main body portion 10 and a state of being close to the main body portion 10, for example.

FIG. 2 is a diagram illustrating a state in which the electrotherapy device according to Embodiment 1 is positioned at the body surface of the user. FIG. 3 is a diagram illustrating a state in which the electrode portion of the electrotherapy device according to Embodiment 1 is attached to the body surface of the user. An operation of attaching the electrotherapy device 100 to the body surface 81 will be described with reference to FIG. 2 and FIG. 3.

As illustrated in FIG. 2, in positioning the electrotherapy device 100 at the body surface 81 of the user 80, the non-adhesive portion 40 is caused to abut the body surface 81 at a desired position. As described above, since the non-adhesive portion 40 protrudes toward the body surface 81 side from the electrode portion 20, the adhesive portion of the electrode portion 20 does not adhere to the body surface 81 in a state in which the non-adhesive portion 40 abuts the body surface 81. Therefore, by moving the non-adhesive portion 40 to the desired position while keeping the non-adhesive portion 40 in contact with the body surface, the electrotherapy device 100 can be easily positioned at an appropriate position.

Subsequently, by operating the operation portion 50 in a state in which the non-adhesive portion 40 abuts the desired position of the body surface 81, the adhesive portion of the electrode portion 20 is caused to abut the body surface 81 as illustrated in FIG. 3. Specifically, the operation portion 50 is operated by pushing the non-adhesive portion 40 toward the body surface 81 via the main body portion 10. The operation portion 50 is provided such that the connection portion connected to the non-adhesive portion 40 is deformable between a state of being away from the main body portion 10 and a state of being close to the main body portion 10. Therefore, when the connection portion of the operation portion 50 is deformed so as to approach the main body portion 10, the non-adhesive portion 40 moves so as to approach the main body portion 10. Accordingly, the non-adhesive portion 40 is positioned farther from the body surface 81 than the adhesive portion of the electrode portion 20, and thus the adhesive portion abuts the body surface 81. As a result, the electrode portion 20 is attached to a desired treatment site of the body surface 81.

As described above, in the electrotherapy device 100 according to Embodiment 1, the adhesive portion provided at the electrode portion 20 can be accurately attached to the desired treatment site with a simple operation.

### Embodiment 2

FIG. 4 is a schematic diagram of an electrotherapy device according to Embodiment 2. In FIG. 4, for convenience, the second electrode portion 22 of the electrode portion 20 and the second holding portion 32 of the holding portion 30 are omitted. The second electrode portion 22 and the second holding portion 32 have substantially the same configuration as the first electrode portion 21 and the first holding portion 31 and exhibit substantially the same behavior as the first electrode portion 21 and the first holding portion 31, and thus the description thereof is also omitted.

As illustrated in FIG. 4, an electrotherapy device 100A according to Embodiment 2 is different from the electrotherapy device 100 according to Embodiment 1 mainly in that the holding portion 30 is movably provided and in the configuration of the operation portion 50, and accordingly, the operation of attaching the electrotherapy device 100A is also different.

As illustrated in FIG. 4, the holding portion 30 is provided so as to be movable in a DR1 direction. The DR1 direction is a direction toward or away from the body surface 81 in a state in which the electrotherapy device 100A faces the body surface 81. The holding portion 30 is held by the main body portion 10 so as to be slidable in the DR1 direction, for example. The holding portion 30 is provided so as not to interfere with the circuit unit.

In the present embodiment, the main body portion 10 includes a bottom surface 10a and a top surface 10b, and functions as the non-adhesive portion 40. The bottom surface 10a faces the body surface 81 side in a facing state in which the electrotherapy device 100A faces the body surface 81. The top surface 10b faces a side opposite to the side on which the body surface 81 is located in the facing state.

The operation portion 50 is provided at the holding portion 30. The operation portion 50 is implemented as a switch 55 such as a button, for example. The switch 55 is provided so as to move integrally with the holding portion 30.

The holding portion 30 is provided in a plate shape. The holding portion 30 includes a first surface 31a and a second surface 31b. The first surface 31a faces the body surface 81 in an abutting state in which the bottom surface 10a as the non-adhesive portion 40 abuts the body surface 81. The second surface 31b is located on a side opposite to the first surface 31a in the abutting state. That is, the second surface 31b faces the side opposite to the side on which the body surface 81 is located in the abutting state.

The first surface 31a is provided with the electrode portion 20. The second surface 31b is provided with the switch 55.

FIG. 5 is a diagram illustrating a state in which the electrotherapy device according to Embodiment 2 is positioned at the body surface of the user. FIG. 6 is a diagram illustrating a state in which the electrode portion of the electrotherapy device according to Embodiment 2 is attached to the body surface of the user. An operation of attaching the electrotherapy device 100A to the body surface 81 will be described with reference to FIG. 5 and FIG. 6.

As illustrated in FIG. 5, in positioning the electrotherapy device 100A at the body surface 81 of the user 80, the bottom surface 10a of the main body portion 10 is caused to abut the body surface 81 at a desired position. The bottom surface 10a is provided so as to protrude toward the body surface 81 side from the electrode portion 20 in a state in which the electrotherapy device 100A is disposed separately from the body surface 81 such that the electrode portion 20 faces the body surface 81.

The adhesive portion of the electrode portion 20 does not adhere to the body surface 81 in a state in which the bottom surface 10a abuts the body surface 81. Therefore, by moving the non-adhesive portion 40 to the desired position while keeping the non-adhesive portion 40 in contact with the body surface, the electrotherapy device 100A can be easily positioned at an appropriate position.

As illustrated in FIG. 6, in Embodiment 2, when the holding portion 30 is moved by operating the switch 55 as the operation portion 50, the adhesive portion of the electrode portion 20 abuts the body surface 81. Specifically, by pushing the switch 55 so as to approach the body surface 81 in a state in which the bottom surface 10a abuts the desired position of the body surface 81, the holding portion 30 moves to approach the body surface 81. Accordingly, the adhesive portion abuts the body surface 81. As a result, the electrode portion 20 is attached to a desired treatment site of the body surface 81.

As described above, also in the electrotherapy device 100A according to Embodiment 2, the adhesive portion provided at the electrode portion 20 can be accurately attached to the desired treatment site with a simple operation.

### Embodiment 3

FIG. 7 is a schematic diagram of an electrotherapy device according to Embodiment 3. In FIG. 7, for convenience, the second electrode portion 22 of the electrode portion 20 and the second holding portion 32 of the holding portion 30 are omitted. The second electrode portion 22 and the second holding portion 32 have substantially the same configuration as the first electrode portion 21 and the first holding portion 31, and exhibit substantially the same behavior as the first electrode portion 21 and the first holding portion 31, and thus the description thereof is also omitted.

As illustrated in FIG. 7, an electrotherapy device 100B according to Embodiment 3 is different from the electrotherapy device 100A according to Embodiment 2 mainly in that the holding portion 30 is fixed to the main body portion 10 and the operation portion 50 is movably provided.

As illustrated in FIG. 7, the switch 55 as the operation portion 50 is provided on the bottom surface 10a side of the main body portion 10, and is provided so as to be movable in the DR1 direction. The switch 55 is held by the main body portion 10 so as to be slidable in the DR1 direction. In the present embodiment, the switch 55 also functions as a non-adhesive portion as will be described below. The holding portion 30 is fixed to the main body portion 10 so as not to be movable. The electrode portion 20 is provided at the first surface 31a of the holding portion 30.

FIG. 8 is a diagram illustrating a state in which the electrotherapy device according to Embodiment 3 is positioned at the body surface of the user. FIG. 9 is a diagram illustrating a state in which the electrode portion of the electrotherapy device according to Embodiment 3 is attached to the body surface of the user. An operation of attaching the electrotherapy device 100B to the body surface 81 will be described with reference to FIG. 8 and FIG. 9.

As illustrated in FIG. 8, in positioning the electrotherapy device 100B at the body surface 81 of the user 80, the switch 55 is caused to abut the body surface 81 at a desired position. The switch 55 is provided so as to protrude toward the body surface 81 side from the electrode portion 20 in a state in which the electrotherapy device 100B is disposed separately from the body surface 81 such that the electrode portion 20 faces the body surface 81.

The adhesive portion of the electrode portion 20 does not adhere to the body surface 81 in a state in which the switch 55 abuts the body surface 81. Therefore, by moving the switch 55 to the desired position while keeping the switch 55 in contact with the body surface, the electrotherapy device 100B can be easily positioned at an appropriate position.

As illustrated in FIG. 9, in Embodiment 3, by pushing the switch 55 as the operation portion 50, the position of the main body portion 10 is changed and the adhesive portion of the electrode portion 20 abuts the body surface 81. Specifically, the switch 55 is pushed toward the body surface 81 in a state in which the switch 55 abuts the body surface 81 at the desired position. Accordingly, the main body portion 10 moves relative to the switch 55 so as to approach the body surface 81. The holding portion 30 moves integrally with the main body portion 10 with the movement of the main body portion 10, and the adhesive portion abuts the body surface 81. As a result, the electrode portion 20 is attached to a desired treatment site of the body surface 81.

In this way, also in the electrotherapy device 100B according to Embodiment 3, the adhesive portion provided at the electrode portion 20 can be accurately attached to the desired treatment site with a simple operation.

### Embodiment 4

FIG. 10 is a schematic diagram of an electrotherapy device according to Embodiment 4. In FIG. 10, for convenience, the second electrode portion 22 of the electrode portion 20 and the second holding portion 32 of the holding portion 30 are omitted. The second electrode portion 22 and the second holding portion 32 have substantially the same configuration as the first electrode portion 21 and the first holding portion 31, and exhibit substantially the same behavior as the first electrode portion 21 and the first holding portion 31, and thus the description thereof is also omitted.

As illustrated in FIG. 10, an electrotherapy device 100C according to Embodiment 4 is different from the electrotherapy device 100 according to Embodiment 1 mainly in the configuration of the operation portion 50 and in that the holding portion 30 is rotatably provided.

In the present embodiment, the main body portion 10 rotatably supports the holding portion 30 and functions as a non-adhesive portion. The first holding portion 31 includes one end 31c and another end 31d. The first holding portion 31 has a rotation center between the one end 31c and the other end 31d. The one end 31c is located on a side opposite to a side on which the second holding portion 32 is located, and the other end 31d is located on the side on which the second holding portion 32 is located.

The electrode portion 20 (the first electrode portion 21) is provided on the one end 31c side, and a lever 56 as the operation portion 50 is provided on the other end 31d side. The first holding portion 31 is supported by the main body portion 10 such that the one end 31c side can be rotated so as to approach and move away from the body surface 81.

FIG. 11 is a diagram illustrating a state in which the electrotherapy device according to Embodiment 4 is positioned at a body surface of a user. FIG. 12 is a diagram illustrating a state in which the electrode portion of the electrotherapy device according to Embodiment 4 is attached to the body surface of the user. An operation of attaching the electrotherapy device 100C to the body surface 81 will be described with reference to FIG. 11 and FIG. 12.

As illustrated in FIG. 11, in positioning the electrotherapy device 100C at the body surface 81 of the user 80, the main body portion 10 (more specifically, the bottom surface 10a) is caused to abut the body surface 81 at a desired position. The lever 56 is located separately from the body surface 81 in an abutting state in which the main body portion 10 abuts the body surface 81. The one end 31c of the holding portion 30 (the first holding portion 31) is located farther from the body surface 81 than the lever 56. Therefore, the adhesive portion of the electrode portion 20 does not adhere to the body surface 81, and the electrotherapy device 100C can be easily positioned at an appropriate position by moving the main body portion 10 to a desired position while keeping the main body portion 10 in contact with the body surface 81.

As illustrated in FIG. 12, from the abutting state, when the lever 56 is moved away from the body surface 81 to rotate the holding portion 30 such that the one end 31c of the first holding portion 31 approaches the body surface 81, the adhesive portion of the electrode portion 20 abuts the body surface 81. As a result, the electrode portion 20 is attached to a desired treatment site of the body surface 81.

In this way, also in the electrotherapy device 100C according to Embodiment 4, the adhesive portion provided at the electrode portion 20 can be accurately attached to the desired treatment site with a simple operation.

### Embodiment 5

FIG. 13 is a schematic diagram of an electrotherapy device according to Embodiment 5. In FIG. 13, for convenience, a control unit 90 is illustrated outside the main body portion 10, but the control unit 90 is provided inside the main body portion 10. Further, in FIG. 13, for convenience, the second electrode portion 22 of the electrode portion 20 and the second holding portion 32 of the holding portion 30 are omitted. The second electrode portion 22 and the second holding portion 32 have substantially the same configuration as the first electrode portion 21 and the first holding portion 31, and thus the description thereof is also omitted.

As illustrated in FIG. 13, an electrotherapy device 100D according to Embodiment 5 is different from the electrotherapy device 100 according to Embodiment 1 mainly in that a drive unit 60 and the control unit 90 are provided and in the configuration of the operation portion 50, and accordingly, the operation of attaching the electrotherapy device 100D is also different.

In the present embodiment, the main body portion 10 functions as a non-adhesive portion. The drive unit 60 and the control unit 90 are disposed in the main body portion 10. The drive unit 60 moves the holding portion 30 while holding the holding portion 30. The drive unit 60 includes, for example, a power generation source such as a motor and a power conversion unit that converts the power of the power generation source into a moving force of the holding portion 30. The control unit 90 is provided at the circuit unit described above. The control unit 90 controls the operation of the drive unit 60.

A switch 57 as the operation portion 50 is provided on the top surface 10b side of the main body portion 10. The switch 57 is for operating the drive unit 60. Specifically, when the switch 57 is pressed, an operation signal is input to the control unit 90, and the control unit 90 controls the operation of the drive unit 60 based on the input operation signal.

FIG. 14 is a diagram illustrating a state in which the electrotherapy device according to Embodiment 5 is positioned at a body surface of a user. FIG. 15 is a diagram illustrating a state in which the electrode portion of the electrotherapy device according to Embodiment 5 is attached to the body surface of the user. As described above, in FIG. 14 and FIG. 15, for convenience, the control unit 90 is illustrated outside the main body portion 10, but the control unit 90 is provided inside the main body portion 10. An operation of attaching the electrotherapy device 100D to the body surface 81 will be described with reference to FIG. 14 and FIG. 15.

As illustrated in FIG. 14, in positioning the electrotherapy device 100D at the body surface 81 of the user 80, the bottom surface 10a of the main body portion 10 is caused to abut the body surface 81 at a desired position. The bottom surface 10a is provided so as to protrude toward the body surface 81 side from the electrode portion 20 in a state in which the electrotherapy device 100D is disposed separately from the body surface 81 such that the electrode portion 20 faces the body surface 81.

The adhesive portion of the electrode portion 20 does not adhere to the body surface 81 in an abutting state in which the bottom surface 10a abuts the body surface 81. Therefore, by moving the main body portion 10 to the desired position while keeping the main body portion 10 in contact with the body surface, the electrotherapy device 100D can be easily positioned at an appropriate position.

As illustrated in FIG. 15, the switch 57 is pressed in the abutting state. Accordingly, an operation signal is input to the control unit 90, and the control unit 90 controls the operation of the drive unit 60 based on the operation signal. Thus, the holding portion 30 holding the electrode portion 20 moves toward the body surface 81. Then, the adhesive portion of the electrode portion 20 abuts the body surface 81. As a result, the electrode portion 20 is attached to a desired treatment site of the body surface 81.

As described above, also in the electrotherapy device 100D according to Embodiment 5, the adhesive portion provided at the electrode portion 20 can be accurately attached to the desired treatment site with a simple operation.

### Embodiment 6

FIG. 16 is a schematic diagram of an electrotherapy device according to Embodiment 6. In FIG. 16, for convenience, the control unit 90 is illustrated outside the main body portion 10, but the control unit 90 is provided inside the main body portion 10. Further, in FIG. 16, for convenience, the second electrode portion 22 of the electrode portion 20 and the second holding portion 32 of the holding portion 30 are omitted. The second electrode portion 22 and the second holding portion 32 have substantially the same configuration as the first electrode portion 21 and the first holding portion 31, and thus the description thereof is also omitted.

As illustrated in FIG. 16, an electrotherapy device 100E according to Embodiment 6 is different from the electrotherapy device 100 according to Embodiment 1 mainly in that the control unit 90, an interposed member 71, and a retracting portion 70 are provided and in the configuration of the operation portion 50.

Also in the present embodiment, the main body portion 10 functions as a non-adhesive portion. The interposed member 71 is provided so as to cover the adhesive portion of the electrode portion 20. The interposed member 71 is a member that is interposed between the adhesive portion and the body surface 81 in an abutting state in which the main body portion 10 (more specifically, the bottom surface 10a) abuts the body surface 81 such that the adhesive portion is not in contact with the body surface 81. The interposed member 71 is formed of a sheet-like member, for example.

The retracting portion 70 is provided so as to be capable of retracting the interposed member 71. The retracting portion 70 is implemented as, for example, a winding roller, and can reel in the interposed member 71. The retracting portion 70 is disposed in the main body portion 10. The operation of the retracting portion 70 is controlled by the control unit 90. The control unit 90 is provided inside the main body portion 10. More specifically, the control unit 90 is provided at the circuit unit described above.

As the operation portion 50, a switch 57 for driving the retracting portion 70 is provided. The retracting portion 70 can be operated by pressing the switch 57. Specifically, when the switch 57 is pressed, an operation signal is input to the control unit 90, and the control unit 90 controls the operation of the retracting portion 70 based on the operation signal. The switch 57 is provided at the top surface 10b of the main body portion 10.

FIG. 17 is a diagram illustrating a state in which the electrotherapy device according to Embodiment 6 is positioned at a body surface of a user. FIG. 18 is a diagram illustrating a state in which the electrode portion of the electrotherapy device according to Embodiment 6 is attached to the body surface of the user. As described above, in FIG. 17 and FIG. 18, for convenience, the control unit 90 is illustrated outside the main body portion 10, but the control unit 90 is provided inside the main body portion 10. An operation of attaching the electrotherapy device 100E to the body surface 81 will be described with reference to FIG. 17 and FIG. 18.

As illustrated in FIG. 17, in positioning the electrotherapy device 100E at the body surface 81 of the user 80, the bottom surface 10a of the main body portion 10 is caused to abut the body surface 81 at a desired position. The bottom surface 10a is located at a position substantially parallel to the adhesive portion of the electrode portion 20, and the interposed member 71 is interposed between the adhesive portion and the body surface 81 as described above.

As illustrated in FIG. 18, in Embodiment 6, the interposed member 71 is retracted by the retracting portion 70 by operating the operation member 50. Specifically, an operation signal is input from the operation portion 50 to the control unit 90, and the control unit 90 drives the retracting portion 70 based on the operation signal. Accordingly, the interposed member 71 is retracted by the retracting portion 70, and the adhesive portion of the electrode portion 20 abuts the body surface 81. As a result, the electrode portion 20 is attached to a desired treatment site of the body surface 81.

As described above, also in the electrotherapy device 100E according to Embodiment 6, the adhesive portion provided at the electrode portion 20 can be accurately attached to the desired treatment site with a simple operation.

Note that, in Embodiment 5, an example in which the retracting portion 70 is controlled by the control unit 90 has been described, but the present disclosure is not limited thereto. The retracting portion 70 may be implemented as a reel portion that is provided so as to be rotatable by an elastic biasing member such as a spiral spring, and the reel portion may be rotated by a biasing force of the elastic biasing member to reel in the interposed member 71 when the switch 57 is pressed.

### Embodiment 7

FIG. 19 is a schematic diagram of an electrotherapy device according to Embodiment 7. In FIG. 19, for convenience, the second electrode portion 22 of the electrode portion 20 and the second holding portion 32 of the holding portion 30 are omitted. The second electrode portion 22 and the second holding portion 32 have substantially the same configuration as the first electrode portion 21 and the first holding portion 31, and thus the description thereof is also omitted.

As illustrated in FIG. 19, an electrotherapy device 100F according to Embodiment 7 is different from the electrotherapy device 100 according to Embodiment 1 mainly in that a lock mechanism 75 is provided and in the configuration of the operation portion 50 and the configuration of the holding portion 30, and accordingly, the operation of attaching the electrotherapy device 100F is also different.

In the present embodiment, the main body portion 10 functions as a non-adhesive portion. The holding portion 30 is provided so as to be elastically deformable. The holding portion 30 is deformed so as to exert an elastic force in a state before the electrode portion 20 is attached to the body surface 81. Specifically, the holding portion 30 is bent in a U-shape from a flat state before deformation.

More specifically, in a state in which the electrotherapy device 100F is opposed to the body surface 81, the one end 31c side on which the electrode portion 20 is provided is bent in a U-shape such that the one end 31c side is located farther from the body surface 81 than the other end 31d is. That is, the holding portion 30 (more specifically, the first holding portion 31) is bent such that a part of the first surface 31a located on the other end 31d side faces the body surface 81 side and a part of the first surface 31a located on the one end 31c side faces a side opposite to the side on which the body surface 81 is located.

The lock mechanism 75 releasably maintains a state in which the holding portion 30 is elastically deformed so as to exert an elastic force. The lock mechanism 75 includes, for example, an engaging portion provided at the one end 31c and an engaged portion provided at the main body portion 10. The engaging portion and the engaged portion are releasably engaged with each other.

The main body portion 10 is provided with a switch 58 as the operation portion 50, and a locked state by the lock mechanism 75 can be released by pressing the switch 58. That is, an engagement state between the engaging portion and the engaged portion can be released.

FIG. 20 is a diagram illustrating a state in which the electrotherapy device according to Embodiment 7 is positioned at a body surface of a user. FIG. 21 is a diagram illustrating a state in which the electrode portion of the electrotherapy device according to Embodiment 7 is attached to the body surface of the user. An operation of attaching the electrotherapy device 100F to the body surface 81 will be described with reference to FIG. 20 and FIG. 21.

As illustrated in FIG. 20, in positioning the electrotherapy device 100F at the body surface 81 of the user 80, the bottom surface 10a of the main body portion 10 is caused to abut the body surface 81 at a desired position. The holding portion 30 and the electrode portion 20 are located separately from the body surface 81 because the holding portion 30 is bent. The adhesive portion of the electrode portion 20 does not adhere to the body surface 81 in an abutting state in which the bottom surface 10a abuts the body surface 81. Therefore, by moving the main body portion 10 to the desired position while keeping the main body portion 10 in contact with the body surface 81, the electrotherapy device 100F can be easily positioned at an appropriate position.

As illustrated in FIG. 21, the switch 58 is pressed in the abutting state. Accordingly, the locked state by the lock mechanism 75 is released, and the holding portion 30 moves by a restoring force based on the elastic force. Specifically, the holding portion 30 is deformed to the original flat state by the restoring force. Then, the adhesive portion of the electrode portion 20 abuts the body surface 81. As a result, the electrode portion 20 is attached to a desired treatment site of the body surface 81.

As described above, also in the electrotherapy device 100F according to Embodiment 7, the adhesive portion provided at the electrode portion 20 can be accurately attached to the desired treatment site with a simple operation.

### Embodiment 8

FIG. 22 is a schematic diagram of an electrotherapy device according to Embodiment 8. An electrotherapy device 100G according to Embodiment 8 will be described with reference to FIG. 22.

As illustrated in FIG. 22, the electrotherapy device 100G according to Embodiment 8 is different from the electrotherapy device 100D according to Embodiment 5 mainly in that an operation terminal 95 provided with an operation input unit 59 as the operation portion 50 is provided. Other configurations are almost the same.

In Embodiment 8, the operation input unit 59 as the operation portion is provided at the operation terminal 95. The operation terminal 95 is provided independently of the main body portion 10. The operation terminal 95 is located separately from the main body portion 10. A user can, for example, grip and move the operation terminal 95. The operation terminal 95 is a remote controller, for example.

The operation terminal 95 includes a transmission unit 96 that transmits an operation signal to the control unit 90. When the operation input unit 59 is operated, the transmission unit 96 transmits an operation signal to the control unit 90. The control unit 90 controls the operation of the drive unit 60 based on the input operation signal.

FIG. 23 is a diagram illustrating a state in which the electrotherapy device according to Embodiment 8 is positioned at a body surface of the user. FIG. 24 is a diagram illustrating a state in which the electrode portion of the electrotherapy device according to Embodiment 8 is attached to the body surface of the user. An operation of attaching the electrotherapy device 100G to the body surface 81 will be described with reference to FIG. 23 and FIG. 24.

As illustrated in FIG. 23, in positioning the electrotherapy device 100G at the body surface 81 of the user 80, the bottom surface 10a of the main body portion 10 is caused to abut the body surface 81 at a desired position. The bottom surface 10a is provided so as to protrude toward the body surface 81 side from the electrode portion 20 in a state in which the electrotherapy device 100G is disposed separately from the body surface 81 such that the electrode portion 20 faces the body surface 81.

The adhesive portion of the electrode portion 20 does not adhere to the body surface 81 in an abutting state in which the bottom surface 10a abuts the body surface 81. Therefore, by moving the main body portion 10 to the desired position while keeping the main body portion 10 in contact with the body surface, the electrotherapy device 100G can be easily positioned at an appropriate position.

As illustrated in FIG. 24, the operation input unit 59 is operated. The operation input unit 59 may be a switch or a touch panel. When the operation input unit 59 is operated, the transmission unit 96 transmits an operation signal to the control unit 90. The control unit 90 controls the operation of the drive unit 60 based on the input operation signal. Thus, the holding portion 30 holding the electrode portion 20 moves toward the body surface 81. Then, the adhesive portion of the electrode portion 20 abuts the body surface 81. As a result, the electrode portion 20 is attached to a desired treatment site of the body surface 81.

As described above, also in the electrotherapy device 100G according to Embodiment 8, the adhesive portion provided at the electrode portion 20 can be accurately attached to the desired treatment site with a simple operation.

Note that, in Embodiments 1 to 8, examples in which the electrode portion 20 includes two electrode portions, that is, the first electrode portion 21 and the second electrode portion 22, and these electrode portions are fixed to one main body portion 10 by the holding portion 30 has been described, but the present disclosure is not limited thereto. There may be a configuration in which two units each including one main body portion 10 provided with one electrode portion 20 are used the holding portion, and an operation portion is operated in each of the two units so as to cause an adhesive portion to abut a body surface.

In addition, in Embodiments 1 to 8, the operation portion 50 may be provided so as to be operable as a power switch of the main body portion 10.

### SUPPLEMENTARY NOTES

As described above, the present embodiment includes the following disclosures.

Configuration 1: An electrotherapy device including: an electrode portion including an adhesive portion; a non-adhesive portion provided so as to be capable of being in contact with a body surface; and an operation portion configured to switch from a state in which the adhesive portion is not in contact with the body surface to a state in which the adhesive portion is in contact with the body surface, wherein the adhesive portion abuts the body surface, by operating the operation portion in an abutting state in which the non-adhesive portion abuts the body surface such that the adhesive portion is not in contact with the body surface.

Configuration 2: The electrotherapy device according to Configuration 1, further including a holding portion that is movably provided and holds the electrode portion, wherein the holding portion moves and the adhesive portion abuts the body surface, by operating the operation portion.

Configuration 3: The electrotherapy device according to Configuration 2, wherein the non-adhesive portion movably supports the holding portion.

Configuration 4: The electrotherapy device according to Configuration 2, wherein the operation portion is coupled to the holding portion, and the holding portion moves and the adhesive portion abuts the body surface, by operating the operation portion.

Configuration 5: The electrotherapy device according to Configuration 4, wherein the holding portion includes: a first surface facing the body surface in the abutting state; and a second surface located on a side opposite to the first surface, the electrode portion is provided at the first surface, and the operation portion is provided at the second surface.

Configuration 6: The electrotherapy device according to Configuration 2, wherein the non-adhesive portion rotatably supports the holding portion, the holding portion includes one end and another end, a rotation center of the holding portion is provided between the one end and the other end, the electrode portion is provided on the one end side of the holding portion, and the operation portion is provided on the other end side of the holding portion, in the abutting state in which the non-adhesive portion abuts the body surface such that the adhesive portion is not in contact with the body surface, the one end side of the holding portion is farther from the body surface than the other end side of the holding portion is, and the adhesive portion abuts the body surface, by rotating the holding portion from the abutting state such that the one end side of the holding portion approaches the body surface.

Configuration 7: The electrotherapy device according to Configuration 2, further including: a drive unit configured to move the holding portion, a control unit configured to control an operation of the drive unit, wherein an operation signal is input to the control unit, by operating the operation portion, and the holding portion moves such that the adhesive portion approaches the body surface, through the control unit controlling the operation of the drive unit based on the operation signal.

Configuration 8: The electrotherapy device according to Configuration 7, further including an operation terminal provided with the operation portion, wherein the operation terminal includes a transmission unit configured to transmit the operation signal to the control unit.

Configuration 9: The electrotherapy device according to Configuration 2, wherein the holding portion is provided so as to be elastically deformable, the electrotherapy device further includes a lock mechanism that releasably maintains a state in which the holding portion is elastically deformed so as to exert an elastic force,

the operation portion is provided so as to be capable of releasing the lock mechanism, and the holding portion is moved by a restoring force based on the elastic force and the adhesive portion abuts the body surface, by releasing the lock mechanism via the operation portion.

Configuration 10: The electrotherapy device according to Configuration 1, further including: an interposed member interposed between the adhesive portion and the body surface in the abutting state; and a retracting portion provided to be capable of retracting the interposed member, wherein the interposed member is retracted by the retracting portion and the adhesive portion abuts the body surface, by operating the operation portion.

Configuration 11: The electrotherapy device according to any one of Configurations 1 to 10, wherein the non-adhesive portion is a main body portion that supplies a current to the electrode portion.

Configuration 12: The electrotherapy device according to Configuration 11, wherein the operation portion is provided so as to be operable as a power switch of the main body portion.

The embodiments disclosed herein are illustrative in all respects and are not intended as limitations. The scope of the present invention is defined by the claims, and all modifications within the meaning and scope equivalent to the claims are intended to be included in the present invention.

### REFERENCE SIGNS

10 Main body portion; 10a Bottom surface; 10b Top surface; 11 Housing case; 20 Electrode portion; 21 First electrode portion; 22 Second electrode portion; 30 Holding portion; 31 First holding portion; 31a First surface; 31b Second surface; 31c One end; 31d Other end; 32 Second holding portion; 40 Non-adhesive portion; 50 Operation portion; 55, 57, 58 Switch; 56 Lever; 60 Drive unit; 70 Retracting portion; 71 Interposed member; 75 Lock mechanism; 80 User; 81 Body surface; 90 Control unit; 95 Operation terminal; 96 Transmission unit; 100, 100A, 100B, 100C, 100D, 100E, 100F, 100G Electrotherapy device.

What is claimed is:

1. An electrotherapy device, comprising:
an electrode portion including an adhesive portion;
a non-adhesive portion provided so as to be capable of being into contact with a body surface; and,
an operation portion configured to switch from a state in which the adhesive portion is not in contact with the body surface to a state in which the adhesive portion is in contact with the body surface, wherein:
the adhesive portion abuts the body surface, by operating the operation portion in an abutting state in which the non-adhesive portion abuts the body surface such that the adhesive portion is not in contact with the body surface; and,
the non-adhesive portion is a main body portion that supplies a current to the electrode portion.

2. The electrotherapy device according to claim 1, further comprising a holding portion that is movably provided and holds the electrode portion, wherein the holding portion moves and the adhesive portion abuts the body surface, by operating the operation portion.

3. The electrotherapy device according to claim 2, wherein the non-adhesive portion movably supports the holding portion.

4. The electrotherapy device according to claim 3, wherein:
the operation portion is coupled to the holding portion; and,
the holding portion moves and the adhesive portion abuts the body surface, by pressing the operation portion.

5. The electrotherapy device according to claim 4, wherein the holding portion includes:
a first surface facing the body surface in the abutting state; and,
a second surface located on a side opposite to the first surface, the electrode portion is provided at the first surface, and the operation portion is provided at the second surface.

6. The electrotherapy device according to claim 2, wherein:
the non-adhesive portion rotatably supports the holding portion;
the holding portion includes one end and another end;
the electrode portion is provided on the one end side of the holding portion; and,
the operation portion is provided on the other end side of the holding portion,
in the abutting state in which the non-adhesive portion abuts the body surface such that the adhesive portion is not in contact with the body surface, the one end side of the holding portion is farther from the body surface than the other end side of the holding portion is; and,
the adhesive portion abuts the body surface, by rotating the holding portion from the abutting state such that the one end side of the holding portion approaches the body surface.

7. The electrotherapy device according to claim 2, further comprising:
a drive unit configured to move the holding portion; and,
a control unit configured to control an operation of the drive unit, wherein:
an operation signal is input to the control unit, by operating the operation portion; and,
the holding portion moves such that the adhesive portion approaches the body surface, through the control unit controlling the operation of the drive unit based on the operation signal.

8. The electrotherapy device according to claim 7, further comprising an operation terminal provided with the operation portion, wherein the operation terminal includes a transmission unit configured to transmit the operation signal to the control unit.

9. The electrotherapy device according to claim 2, wherein:
the holding portion is provided so as to be elastically deformable;
the electrotherapy device further comprises a lock mechanism that releasably maintains a state in which the holding portion is elastically deformed so as to exert an elastic force;
the operation portion is provided so as to be capable of releasing the lock mechanism; and,
the holding portion is moved by a restoring force based on the elastic force and the adhesive portion abuts the body surface, by releasing the lock mechanism releasing via the operation portion.

10. The electrotherapy device according to claim 1, further comprising:
an interposed member interposed between the adhesive portion and the body surface in the abutting state; and,
a retracting portion provided to be capable of retracting the interposed member, wherein the interposed member is retracted by the retracting portion and the adhesive portion abuts the body surface, by operating the operation portion.

11. The electrotherapy device according to claim 1, wherein the operation portion is provided so as to be operable as a power switch of the main body portion.

12. The electrotherapy device according to claim 2, wherein the operation portion is provided so as to be operable as a power switch of the main body portion.

13. The electrotherapy device according to claim 3, wherein the operation portion is provided so as to be operable as a power switch of the main body portion.

14. The electrotherapy device according to claim 4, wherein the operation portion is provided so as to be operable as a power switch of the main body portion.

15. The electrotherapy device according to claim 5, wherein the operation portion is provided so as to be operable as a power switch of the main body portion.

16. The electrotherapy device according to claim 6, wherein the operation portion is provided so as to be operable as a power switch of the main body portion.

17. The electrotherapy device according to claim 7, wherein the operation portion is provided so as to be operable as a power switch of the main body portion.

18. The electrotherapy device according to claim 8, wherein the operation portion is provided so as to be operable as a power switch of the main body portion.

19. The electrotherapy device according to claim 9, wherein the operation portion is provided so as to be operable as a power switch of the main body portion.

20. The electrotherapy device according to claim 10, wherein the operation portion is provided so as to be operable as a power switch of the main body portion.
